(19) United States
(12) Patent Application Publication
(10) Pub. No.: US 2007/0111954 A1
(43) Pub. Date: May 17, 2007

(54) Apparatus and method of wart treatment

(75) Inventors: Charles E. Crutchfield III, St. Paul, MN (US); Humberto Gallego, Seattle, WA (US)

Correspondence Address: BRIGGS AND MORGAN P.A., 2200 IDS CENTER, 80 SOUTH 8TH ST, MINNEAPOLIS, MN, 55402, US

(73) Assignee: Cuticeuticals, Inc., St. Paul, MN (US)

(21) Appl. No.: 11/586,200

(22) Filed: Oct. 25, 2006

Related U.S. Application Data
(63) Continuation-in-part of application No. 10/625,930, filed on Jul. 23, 2003, now abandoned.

(51) Int. Cl. A61K 31/7048; A61K 31/365; A61K 31/60

(52) U.S. Cl. 514/27; 514/159; 514/468; 604/500

(57) ABSTRACT

A single-use, delivery vehicle for the treatment of warts and molluscum contagiosum, consisting of a solution of cantharidin, salicylic acid, and podophyllin packaged in a crushable ampoule with a soft, absorbent, applicator tip. A method of delivering wart-removing and molluscum removing compositions to the skin, consisting of the steps of crushing an ampoule containing the wart-removing composition and pressing the absorbent tip against the wart or molluscum.

### CROSS-REFERENCE TO RELATED APPLICATIONS

The present application is a Continuation-In-Part of and claims priority of U.S. aplication Ser. No. 10/625,930, filed Jul. 23, 2003 entitled WART TREATMENT.

### BACKGROUND OF THE INVENTION

The present invention relates to a single-use, delivery vehicle for treatment of cutaneous warts.

Cutaneous warts (verrucae) are benign, epithelial tumors characterized by the formation of thick, hyperkaratotic lesions. Human papillomavirus (HPV) is a double-stranded DNA virus, and is responsible for the appearance of warts. Virus particles reside in the basal layer of epithelia, but replicate only in the well-differentiated, superficial layer. The ensuing cellular proliferation gives rise to the characteristic morphology of warts. Several types of cutaneous warts have been described: common warts (verrucae vulgaris), plantar warts (verrucae plantaris),and flat warts (verrucae planus). Although verrucae are more commonly encountered in children and young adults, all ages may be affected. At particular risk are immunocompromised individuals. Humal papillomavirus may be transmitted indirectly through contact with the skin of an infected individual or by transmission of virus that has survived in warm, moist environments. The virus may also be transferred from one site to another when autoinoculation occurs upon traumatizing warts by scratching or biting. The incubation period is unknown, but may be several months or years.

Molluscum contagiosum is a disease caused by a poxvirus of the Molluscipox virus genus that produces a benign self-limited papular eruption of multiple umbilicated cutaneous tumors. This common viral disease is confined to the skin and mucous membranes. Transmission requires direct contact with infected hosts or contaminated fomites. It is generally thought to infect humans exclusively, but there are a few isolated reports of Molluscum contagiosum occurring in chickens, sparrows, pigeons, chimpanzees, kangaroos, a dog, and a horse. The infection is found worldwide and has a higher incidence in children, sexually active adults, and those who are immunodeficient.

Several treatment modalities for warts have been used in the past, but none have been universally successful. Chemotherapy, cryotherapy, surgery, immunotherapy, and other treatment methods have been attempted.

Treatment modalities for molluscum contagiosum have included cryosurgery, evisceration, curettage, tape stripping, and chemotherapy.

In the area of chemotherapy, cantharidin has been used for treatment of warts and molluscum. Cantharidin is derived from the blister beetle (Coleopteres heteromeres) and poisons mitochondria, leading to acantholysis and vesicle formation. Plantar and periungual warts are commonly treated with cantharidin, typically as a 0.7% collodion solution after paring the wart with a blade. Because warts are deep, the mixture at 0.7% to 1% is left on for anywhere from an hour to several hours (over night) so that it can work its way deep into the wart and cause a blister that will be deep enough to take off a significant portion of the wart.

Mollluscum is different. These bumps are smaller and do not go deep. Therefore, the cantharidin is left on for anywhere from about 10 minutes to a couple of hours and then washed off. This should result in a shallow blister that takes off the molluscum bump without causing too much damage to the skin. If the cantharadin is left on for too long, it can result in severe injury, pain, and scarring. Therefore, it is somewhat tricky to use cantharadin in treating molluscum and one has to be very careful not to do harm. At the same time, if one washes it off too early, it does not work.

Podophyllin may also be used as a chemotherapeutic agent. It is derived from the Podophyllum peltatum and p emodi plant species. It interrupts cellular activity at metaphase as it combines with tubulin. It also exerts its effect on verrucae by disrupting the microcirculation. Podophyllin is typically applied in a 25% liquid paraffin base or 0.5% podophyllotoxin in alcohol to the debrided wart surface. Cantharidin, alone or in combination with podophyllin, was commercially available until 1991 when the FDA ruled that salicylic acid could be obtained without a prescription, whereas other agents (cantharidin included) could not be marketed until safety and efficacy issues were resolved.

In typical application methods, cantharidin and podophyllin are dissolved in acetone and stored in single-use, multi-dose bottles. Accordingly, the composition has a tendency for the acetone to evaporate when the bottle is opened, leading to drying out. In addition, because of large amount of composition compared to the small amount needed per dose, practitioners have a tendency to use the same bottle for more than one patient. This can lead to unwanted cross-contamination. These application methods also do not provide precise control of delivery to the wart.

Single-use applicators are known for the delivery of various composition to treat warts. However, such single-use applicators are typically complex, requiring moving parts to crush the ampoule, causing increased cost of manufacture. It is not known to the applicant whether single-use applicators have been used to treat molluscum.

Johnson (U.S. Pat. No. 5,445,462) is typical of previous applicators. Johnson requires a complex set of moving parts to crush the ampoule and also including a locking means to prevent movement of the crushing element from the storage position to the use position or vice-versa. This complexity increases the cost of manufacture.

Strauss (U.S. Pat. No. 6,503,013) uses a swab to apply medicament. This would entail contaminating the skin surface adjacent to the wart or molluscum, which should be avoided.

There is a need for an improved wart-removing composition and for an improved, single-use delivery vehicle.

There is also a need for an improved composition that can safely remove molluscum. The cantharadin is present in a very small concentration (0.035-0.1%). At this low concentration, blistering is unlikely to occur. However, cantharadin can still cause skin irritation at this concentration. Skin irritation can result in clearing the molluscum without the need or risks of blistering. The cantharadin can simply be left on without worrying about when to wash it off. This would make cantharadin much easier to use in the treatment of molluscum.

### SUMMARY OF THE INVENTION

A single-use, delivery vehicle for the treatment of warts and molluscum contagiousum, consisting of a solution of cantharidin, salicylic acid and podophyllin packaged in a crushable ampoule with a soft, absorbent, applicator tip.

A method of delivering wart-removing compositions to the skin, consisting of the steps of crushing an ampoule containing the wart-removing composition and pressing the absorbent tip against the wart.

A principle object and advantage of the present invention is that it prevents the wart-removing composition from drying out.

Another principle object and advantage of the present invention is that cross-contamination between patients is prevented.

Another principle object and advantage of the present invention is that it minimizes waste.

Another principle object and advantage of the present invention is that it provides precise control of delivery of the composition.

Another principal object and advantage of the present invention is that it can be used for treating molluscum contagiosum.

Another principle object and advantage of the present invention is that no moving parts are needed to crush the ampoule, thus decreasing the cost of manufacture.

### BRIEF DESCRIPTION OF THE DRAWINGS

FIG. 1 is a schematic of the single-use, delivery vehicle of the present invention.

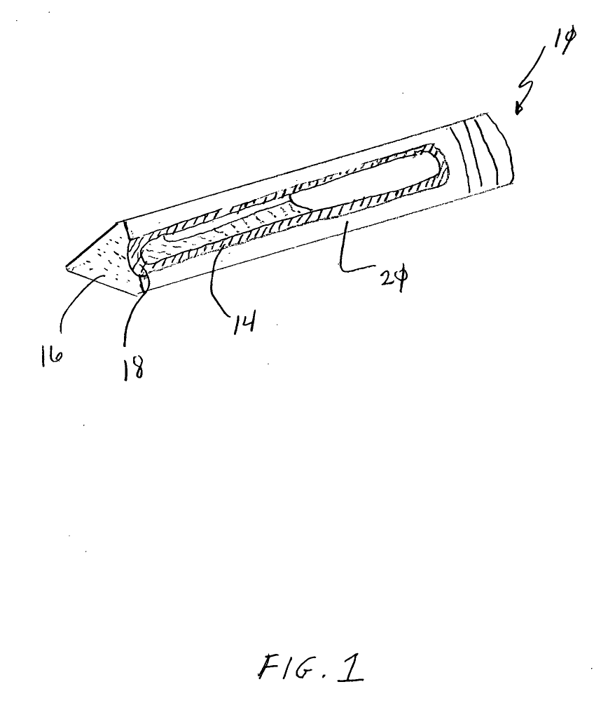

### DETAILED DESCRIPTION OF THE PREFERRED EMBODIMENT

The single-use, delivery vehicle for the treatment of cutaneous warts of the present invention is generally shown in the Figures as reference numeral 10.

The single-use, delivery vehicle 10 of the present invention comprises a solution 12 of cantharidin, salicylic acid, and podophyllin.

Preferably, the single-use, delivery vehicle 10 further comprises a crushable ampoule 14 in which the solution 12 is packaged. The single-use, delivery vehicle further preferably comprises a soft, absorbent, applicator tip 16 separated from the crushable ampoule 14 by a wall 18. When the ampoule 14, is crushed, the wall 18 is disrupted, causing the solution 12 to flow to the applicator tip 16. The applicator tip 16 is tapered, allowing very precise application of the composition.

Preferably, the single-use, delivery vehicle 10 further comprises a flexible outer wall 20 that protects the crushable ampoule 14 and is squeezed to crush the ampoule 14. Thus, no moving parts are needed to crush the ampoule.

In one embodiment, used to treat warts, the cantharidin may preferably be present in the solution 12 at about 0.5% to about 1.5% by weight and most preferably at about 1% by weight.

In a second embodiment, used to treat molluscum, the cantharadin is preferably present at about 0.035% to about 0.1% by weight.

The solution 12 may also further comprise salicylic acid at about 12% to about 40% by weight, most preferably at about 30% by weight.

Preferably, the podophyllin is present at about 2.5% to about 20% by weight, most preferably at about 5% by weight.

The solution 12 may also further comprise acetone at about 30% to about 50% by weight, most preferably at about 40% by weight.

The solution 12 may also further comprise tincture of benzoin.

To use the single-use, delivery vehicle 10, the user crushes the ampoule 14 by squeezing the flexible outer wall 20, disrupting the wall 18 and allowing the solution 12 to flow to the applicator tip 16. The applicator tip 16 is then pressed against the wart. Because the applicator tip is tapered, only the wart, and not surrounding skin, receives the solution.

It will be seen that the ampoule is crushed without the need for any moving parts, making the invention much simpler than previous applicators.

The present invention may be embodied in other specific forms without departing from the spirit or essential attributes thereof, and it is therefore desired that the present embodiment be considered in all respects as illustrative and not restrictive, reference being made to the appended claims rather than to the foregoing description to indicate the scope of the invention.

What is claimed:

1. A single-use, delivery vehicle for the treatment of cutaneous warts and molluscum contagiosum, comprising: a solution of: cantharidin, salicylic acid, and podophyllin packaged in a single-use, crushable ampoule with a soft, absorbent applicator tip.

2. The single-use, delivery vehicle of claim 1, the solution comprising podophyllin in about 2.5% to about 20% by weight.

3. The single-use, delivery vehicle of claim 2, wherein the podophyllin is present at about 5% by weight.

4. The single-use, delivery vehicle of claim 1, wherein the cantharidin is present at about 0.5% to 1.5% by weight.

5. The single-use, delivery vehicle of claim 4, wherein the cantharidin is present at about 1% by weight.

6. The single-use, delivery vehicle of claim 1, wherein the cantharadin is present at about 0.035% to about 0.1% by weight.

7. The single-use, delivery vehicle of claim 1, wherein the salicylic acid is present at about 12% to about 40% by weight.

8. The single-use, delivery vehicle of claim 7, wherein the salicylic acid is present at about 30% by weight.

9. The single-use, delivery vehicle of claim 1, further comprising acetone at about 30% to about 50% by weight.

10. The single-use, delivery vehicle of claim 9 wherein the acetone is present at about 40% by weight.

11. The single-use, delivery vehicle of claim 1, the solution further comprising tincture of benzoin.

12. The single-use, delivery vehicle of claim 1, having no moving parts for crushing the ampoule.

13. A method of delivering wart-removing and molluscom-removing compositions to the skin, the composition being packaged in a single-use, crushable ampoule with a soft, absorbent tip, comprising the steps of:
(a) crushing the ampoule, the ampoule containing a wart-removing or molluscum-removing composition; and
(b) pressing the absorbent tip of the ampoule against the wart or molluscum.

14. The method of claim 13, where the wart-removing or mulluscom-removing composition further comprises a solution of cantharidin, salicylic acid, and podophyllin.

15. The method of claim 14, wherein the solution further comprises about 0.5% to about 1.5% by weight cantharidin.

16. The method of claim 15, wherein the solution further comprises about 0.5% by weight cantharidin.

17. The single-use, delivery vehicle of claim 14, wherein the cantharadin is present at about 0.035% to about 0.1% by weight.

18. The method of claim 14, wherein the solution further comprises about 2.5% to about 20% by weight podophyllin.

19. The method of claim 18, wherein the solution further comprises about 5% by weight podophyllin.

20. The method of claim 14, wherein the solution further comprises about 12.5% to about 40% by weight salicylic acid.

21. The method of claim 20, wherein the solution further comprises about 30% by weight salicylic acid.

22. The method of claim 14, wherein the solution further comprises acetone at about 30% to about 50% by weight.

23. The method of claim 22, wherein the solution further comprises about 40% acetone.

24. The method of claim 14, wherein the solution further comprises tincture of benzoin.

25. The method of claim 14, wherein the ampoule is crushed without moving parts.

26. A single-use, delivery vehicle for the treatment of cutaneous warts and molluscum contagiosum, comprising a solution of: cantharidin, salicylic acid, and podophyllin packaged in a single-use, crushable ampoule, the delivery vehicle further comprising a flexible outer wall that protects the crushable ampoule and is squeezed to crush the ampoule, a soft, absorbent, tapered applicator tip, and a disruptable inner wall that separates the solution from the applicator tip.
